(19) United States
(12) Patent Application Publication
(10) Pub. No.: US 2026/0041388 A1
(43) Pub. Date: Feb. 12, 2026

(54) METHOD FOR MEDICAL IMAGING AND MEDICAL IMAGING SYSTEM

(71) Applicant: GE Precision Healthcare LLC, Waukesha, WI (US)

(72) Inventors: Xueli Wang, Beijing (CN); Fengwei Ju, Beijing (CN); Qingfeng Zhang, Beijing (CN)

(21) Appl. No.: 19/295,309

(22) Filed: Aug. 8, 2025

(30) Foreign Application Priority Data
Aug. 8, 2024 (CN) 202411088486.9

(51) Int. Cl. A61B 6/00; A61B 6/58

(52) U.S. Cl. CPC A61B 6/488; A61B 6/582

(57) ABSTRACT

The present disclosure relates to a method for imaging and an imaging system. The method includes obtaining a scanning protocol for an examination subject, which includes: obtaining calibration data associated with the scanning protocol in response to a commonly used scanning protocol calibration data set of a medical imaging system including the calibration data; or performing a calibration operation in response to a commonly used scanning protocol calibration data set of a medical imaging system not including calibration data associated with the scanning protocol, to generate calibration data associated with the scanning protocol. The method further includes scanning the examination subject based on the scanning protocol, to obtain raw imaging data, and performing reconstruction using the calibration data associated with the scanning protocol and the raw imaging data, to obtain a medical image of a scanned subject.

### CROSS-REFERENCE TO RELATED APPLICATIONS

This application claims priority to Chinese Application No. 202411088486.9, filed on Aug. 8, 2024, the disclosure of which is incorporated herein by reference in its entirety.

### TECHNICAL FIELD

The present disclosure relates to the field of imaging, and in particular, to a method for medical imaging, a imaging system, a computer-readable storage medium, and a computer program product.

### BACKGROUND

Medical imaging devices are used for obtaining an anatomical structure of a scanned subject, and include devices that utilize X-rays to perform medical imaging, e.g., Computed Tomography (CT), digital X-ray machines, C-arm X-ray machines, digital subtraction angiography X-ray machines, mammography X-ray machines, etc. For example, when a CT apparatus performs a scan, an X-ray tube emits an X-ray. The X-ray passes through a test object and attenuates. A detector receives the attenuated X-ray, and converts the same into an electrical signal. After a series of processing, a computer reconstructs medical tomographic images for diagnostic reference.

After the medical imaging devices are used for a period of time, the X-ray tube needs to be calibrated. Calibration of the X-ray tube is intended to generate calibration data based on a preset scanning condition, and the calibration data is used in a subsequent medical image reconstruction process. Currently, a commonly used X-ray tube calibration method is to perform exposure detection under all preset scanning conditions. In the X-ray tube calibration method, a large amount of exposure needs to be performed. Therefore, a long time is required to perform detection under respective scanning conditions, and the large amount of exposure affects the service life of the X-ray tube.

### SUMMARY OF THE INVENTION

The present disclosure aims to overcome the above and/or other problems in the prior art, and provides a method for medical imaging, to ensure good imaging quality while reducing a quantity of exposure detection times required for calibration operations, and help prolong the service life of the X-ray tube and improve the use efficiency of the medical imaging system.

According to a first aspect of the present disclosure, a method for imaging is provided. The method includes the steps of obtaining a scanning protocol for an examination subject; for the scanning protocol: obtaining calibration data associated with the scanning protocol in response to a commonly used scanning protocol calibration data set of a medical imaging system comprising the calibration data; or performing a calibration operation in response to a commonly used scanning protocol calibration data set of the medical imaging system not comprising calibration data associated with the scanning protocol, to generate calibration data associated with the scanning protocol; scanning the examination subject based on the scanning protocol, to obtain raw imaging data; and performing reconstruction using the calibration data associated with the scanning protocol and the raw imaging data, to obtain a medical image of the scanned subject.

Optionally, the method further includes obtaining the calibration data in response to a timestamp of the calibration data meeting a first preset condition.

Optionally, the method further includes performing a calibration operation in response to the timestamp of the calibration data not meeting the first preset condition, to update the calibration data associated with the scanning protocol.

Optionally, the calibration operation is automatically performed by the medical imaging system.

Optionally, the calibration operation is performed based on reception of an instruction to perform calibration, and the method further includes providing prompt information about whether to perform calibration.

Optionally, the calibration operation includes generating a calibration parameter for the scanning protocol; scanning air using the calibration parameter, to obtain air imaging data; and determining the calibration data based on the air imaging data.

Optionally, the method further includes updating the calibration data determined based on the air imaging data, a calibration parameter associated with the calibration data, and a new timestamp in the commonly used scanning protocol calibration data set.

Optionally, the commonly used scanning protocol calibration data set includes a correspondence between different calibration parameters and calibration data thereof; and determining whether the calibration data associated with the scanning protocol exists in the commonly used scanning protocol calibration data set comprises: determining a calibration parameter based on the scanning protocol; and determining, based on the calibration parameter, whether the calibration data associated with the calibration parameter exists in the commonly used scanning protocol calibration data set.

Optionally, the commonly used scanning protocol calibration data set is generated via the following steps: obtaining N scanning protocols, wherein N is an integer greater than 1; selecting M scanning protocols from among the N scanning protocols, and determining a plurality of groups of calibration parameters corresponding to the M scanning protocols, wherein N is greater than M, and M is an integer greater than 0; obtaining calibration data associated with each of the plurality of groups of calibration parameters; and storing each of the plurality of groups of calibration parameters and the calibration data associated with each group of calibration parameters in the commonly used scanning protocol calibration data set.

Optionally, the selecting M scanning protocols from among the N scanning protocols includes determining at least a part of the M scanning protocols based on the number of times of use of a plurality of scanning protocols corresponding to a plurality of times of scanning and imaging performed in a period of time.

Optionally, the selecting M scanning protocols from among the N scanning protocols includes determining at least a part of the M scanning protocols based on a plurality of scanning protocols corresponding to a type of a medical institution.

Optionally, the obtaining calibration data associated with each of the plurality of groups of calibration parameters includes for each group of calibration parameters, obtaining default calibration data associated with the group of calibration parameters.

Optionally, the obtaining calibration data associated with each of the plurality of groups of calibration parameters includes for each group of calibration parameters, performing a calibration operation, to generate calibration data associated with the group of calibration parameters.

Optionally, air is scanned using the group of calibration parameters, to obtain air imaging data; and the calibration data associated with the group of calibration parameters is determined based on the air imaging data.

Optionally, the calibration parameter comprises at least one of the following: a tube voltage, a collimator aperture width, a X-ray shape filter size, and a focal spot size.

Optionally, the first preset condition comprises an interval between the timestamp and a current time being less than or equal to a first threshold.

Optionally, the method further includes deleting from the commonly used scanning protocol calibration data set calibration data of which the timestamp meets a second preset condition.

Optionally, the method further includes deleting from the commonly used scanning protocol calibration data set calibration data of which the number of times of use is less than a second threshold.

Optionally, the second preset condition comprises an interval between the timestamp and a current time being greater than or equal to a third threshold.

Optionally, the method further includes determining whether the quality of the medical image is acceptable; performing a calibration operation in response to determining that the quality of the medical image is unacceptable, to update calibration data associated with the calibration parameter; and performing reconstruction using the updated calibration data and the raw imaging data, to obtain an updated medical image.

Optionally, the calibration operation includes generating a calibration parameter for the scanning protocol; scanning air using the calibration parameter, to obtain air imaging data; and updating the calibration data based on the air imaging data.

Optionally, the method further includes updating the updated calibration data, a calibration parameter associated with the updated calibration data, and a new timestamp in the commonly used scanning protocol calibration data set.

According to a second aspect of the present disclosure, an imaging system is provided, including an imaging device, configured to perform scanning and imaging on a scanned subject; and a computer, configured to perform the method described above.

According to a third aspect of the present disclosure, a computer-readable storage medium having a computer program stored thereon is provided, wherein the program, when executed by a processor, implements the steps of the method described above.

According to a fourth aspect of the present disclosure, a computer program product is provided, comprising instructions, wherein the instructions are capable of being executed by a processor, to implement the method described above.

### BRIEF DESCRIPTION OF THE DRAWINGS

The present disclosure can be better understood by means of the description of the exemplary embodiments of the present disclosure in conjunction with the drawings, in which:

FIG. 1 shows an exemplary CT imaging system.

FIG. 2 shows an exemplary imaging system similar to the CT imaging system in FIG. 1.

FIG. 3 shows a schematic flowchart of a method for medical imaging according to an exemplary embodiment of the present disclosure.

FIG. 4 shows a schematic flowchart of a method for medical imaging according to an optional embodiment of the present disclosure.

FIG. 5 shows a schematic process of a calibration operation according to an optional embodiment of the present disclosure.

FIG. 6 shows an example of a commonly used scanning protocol calibration data set according to the present disclosure.

FIG. 7 shows a schematic process of determining whether calibration data exists in a commonly used scanning protocol calibration data set according to the present disclosure.

FIG. 8 shows a schematic process of establishing a commonly used scanning protocol calibration data set according to the present disclosure.

FIG. 9 shows a schematic diagram of selecting a scanning protocol subset from a full scanning protocol set.

FIG. 10 shows optional steps of a method for medical imaging according to an embodiment of the present disclosure.

FIG. 11 shows an example of an electronic device according to an embodiment of the present disclosure.

### DETAILED DESCRIPTION

Specific embodiments of the present disclosure will be described below, but it should be noted that in the specific description of these embodiments, for the sake of brevity of description, it is impossible to describe all features of the actual embodiments of the present disclosure in detail in this description. It should be understood that in the actual implementation process of any implementation, just as in the process of any one engineering project or design project, a variety of specific decisions are often made to achieve specific goals of the developer and to meet system-related or business-related constraints, which may also vary from one implementation to another. Furthermore, it should also be understood that although efforts made in such development processes may be complex and tedious, for those of ordinary skill in the art related to the content of the present disclosure, some design, manufacture, or production changes made on the basis of the technical content disclosed in the present disclosure are only common technical means, and should not be construed as the content of the present disclosure being insufficient.

Unless otherwise defined, the technical or scientific terms used in the claims and the description should be as they are usually understood by those possessing ordinary skill in the technical field to which they belong. The terms “first,” “second,” and the like used in the description and claims of the patent application of the present disclosure do not denote any order, quantity, or importance, but are merely intended to distinguish between different constituents. The terms “one,” “a/an,” and the like do not denote a limitation of quantity, but rather denote the presence of at least one of the referenced items. The terms “include,” “comprise,” and the like are intended to mean that an element or article that appears before “include” or “comprise” encompasses elements or articles and equivalent elements that are listed after “include” or “comprise,” and do not exclude other elements or articles. The terms “connect,” “connected,” and the like are not limited to physical or mechanical connection, and are not limited to direct or indirect connection. “Examination subject” generally includes, but is not limited to, a patient, an animal, or other subjects examined by a medical imaging device.

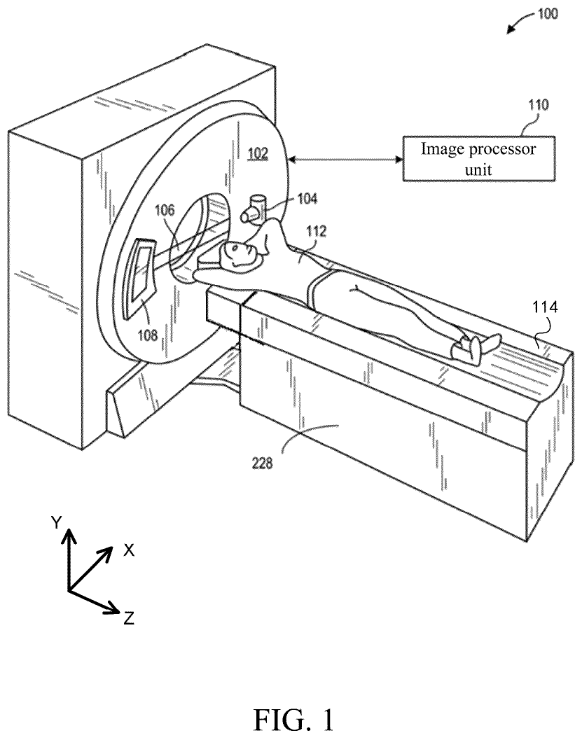

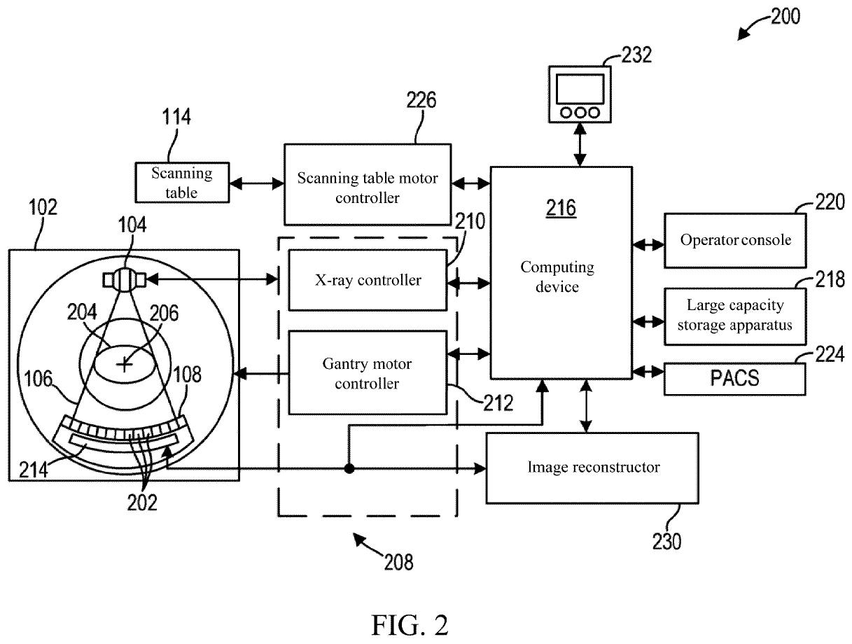

FIG. 1 and FIG. 2 show exemplary embodiments of an imaging system. A method for medical imaging provided by an embodiment of the present disclosure may be applied to the imaging system. While a CT system is described by way of example, it should be understood that the present technique may also be applied to other imaging modalities, such as an X-ray imaging system, a magnetic resonance imaging (MRI) system, a nuclear medical imaging system, a positron emission tomography (PET) imaging system, a single photon emission computed tomography (SPECT) imaging system, an ultrasonic imaging system, and combinations thereof (e.g., a multi-modal imaging system such as a PET/CT or PET/MR imaging system). The discussion on CT imaging modalities in the present disclosure is provided only as an example of one suitable imaging modality.

FIG. 1 shows an exemplary CT imaging system 100 configured for CT imaging. Specifically, the CT imaging system 100 is configured to image an examination subject 112 (such as a patient, an inanimate subject, or one or more manufactured components) and/or a foreign subject (such as a dental implant, a stent, and/or a contrast agent present in the body). In one embodiment, the CT imaging system 100 includes a gantry 102, which in turn may further include at least one X-ray source 104. The at least one X-ray source is configured to project an X-ray radiation beam 106 for imaging the examination subject 112 lying on a scanning table 114. Specifically, the X-ray source 104 is configured to project the X-ray radiation beam 106 toward a detector array 108 positioned on the opposite side of the gantry 102. Although FIG. 1 depicts only a single X-ray source 104, in certain implementations, a plurality of X-ray sources and detectors may be used to project a plurality of X-ray radiation beams 106, so as to acquire projection data corresponding to the patient at different energy levels. In some implementations, the X-ray source 104 may achieve dual-energy gemstone spectral imaging (GSI) by means of rapid peak kilovoltage (kVp) switching. In some implementations, the X-ray detectors which are used are photon counting detectors capable of distinguishing X-ray photons of different energies. In other implementations, dual-energy projections are generated using two sets of X-ray sources and detectors, wherein one set of X-ray sources and detectors is set to low kVp and the other set is set to high kVp. It should therefore be understood that the methods described herein may be implemented using single-energy acquisition techniques and dual-energy acquisition techniques.

In some embodiments, the CT imaging system 100 further includes an image processor unit 110, which is configured to reconstruct an image of a target volume of the examination subject 112 by using an iterative or analytical image reconstruction method. For example, the image processor unit 110 may reconstruct an image of a target volume of the patient using an analytical image reconstruction method such as filtered back projection (FBP). As another example, the image processor unit 110 may reconstruct the image of the target volume of the examination subject 112 in an iterative image reconstruction method (e.g., advanced statistical iterative reconstruction (ASIR), conjugate gradient (CG), maximum likelihood expectation maximization (MLEM), model-based iterative reconstruction (MBIR), etc.). As further described herein, in some examples, in addition to the iterative image reconstruction method, the image processor unit 110 may use an analytical image reconstruction method (such as FBP).

In some CT imaging system configurations, the X-ray source projects a conical X-ray radiation beam, which is collimated to be located within an X-Y-Z plane of a Cartesian coordinate system, and the plane is usually referred to as an “imaging plane”. The X-ray radiation beam passes through a subject being imaged, e.g., a patient or an examination subject. The X-ray radiation beam is irradiated on a detector element array after being attenuated by the subject. The intensity of the attenuated X-ray radiation beam received at the detector array depends on the attenuation of the radiation beam by the subject. Each detector element of the array produces a separate electrical signal that is a measure of the X-ray beam attenuation at the detector position. Attenuation measurements from all detector elements are individually acquired to generate a transmission profile.

In some CT imaging systems, a gantry is used to rotate the X-ray source and the detector array in the imaging plane around the subject to be imaged so that the angle at which the radiation beam intersects the subject is constantly changing. A set of X-ray radiation attenuation measurement results (e.g., projection data) from the detector array at one gantry angle is referred to as a “view”. A “scan” of the subject includes a set of views made at different gantry angles or viewing angles during one rotation of the X-ray source and detector. It can be contemplated that benefits of the method in this specification derive from a medical imaging modality other than CT. Therefore, as used herein, the term “view” is not limited to the use described above with respect to projection data from one gantry angle. The term “view” is used to mean one data acquisition when there are a plurality of data acquisitions (acquisitions from CT, positron emission tomography (PET), or single photon emission CT (SPECT)) from different angles, and/or any other modality (including a modality to be developed) and combinations thereof in fused embodiments.

Projection data is processed to reconstruct images corresponding to two-dimensional slices acquired by means of the subject, or in some examples in which the projection data includes a plurality of views or scans, reconstruct the images corresponding to three-dimensional rendering of the subject. A method for reconstructing an image from a set of projection data is referred to as a filtered back projection technique in the art. Transmission and emission tomography reconstruction techniques also include statistical iterative methods, such as maximum likelihood expectation maximization (MLEM) and ordered subset expectation reconstruction techniques, as well as iterative reconstruction techniques. The method converts an attenuation measurement from a scan into an integer referred to as a “CT number” or “Hounsfield unit”, which is used to control the brightness of a corresponding pixel on a display device.

To reduce the total scan time, a “helical” scan may be performed. To perform the “helical” scan, the patient is moved when data of a specified number of slices is acquired. Such systems produce a single helix from helical scanning of a conical beam. The helix mapped out by the conical beam produces projection data according to which an image in each specified slice can be reconstructed.

As used herein, the phrase “reconstructed image” is not intended to exclude embodiments of the present disclosure in which data representing an image is generated rather than a viewable image. Thus, as used herein, the term “image” broadly refers to both a viewable image and data representing a viewable image. However, many embodiments generate (or are configured to generate) at least one viewable image.

FIG. 2 shows an exemplary imaging system 200 similar to the CT imaging system 100 in FIG. 1. According to aspects of the present disclosure, the imaging system 200 is configured to image the examination subject 204 (e.g., the examination subject 112 in FIG. 1). In one embodiment, the imaging system 200 includes the detector array 108 (see FIG. 1). The detector array 108 further includes a plurality of detector elements 202, which together sense the X-ray radiation beam 106 (see FIG. 2) passing through the examination subject 204 (such as a patient) to acquire corresponding projection data. Therefore, in one embodiment, the detector array 108 is fabricated in a multi-slice configuration including a plurality of rows of units or detector elements 202. In such configurations, one or more additional rows of detector elements 202 are arranged in a parallel configuration for acquiring projection data.

In certain implementations, the imaging system 200 is configured to traverse different angular positions around the examination subject 204 to acquire required projection data. Therefore, the gantry 102 and components mounted thereon can be configured to rotate about a center of rotation 206 to acquire projection data at different energy levels, for example. Alternatively, in implementations in which a projection angle with respect to the examination subject 204 changes over time, the mounted components may be configured to move along a generally curved line rather than a segment of a circumference.

Therefore, when the X-ray source 104 and the detector array 108 rotate, the detector array 108 collects the data of the attenuated X-ray beam. The data collected by the detector array 108 is then subjected to pre-processing and calibration to adjust the data so as to represent a line integral of an attenuation coefficient of the scanned examination subject 204. The processed data is generally referred to as projection data.

In some examples, an individual detector or detector element 202 in the detector array 108 may include a photon counting detector that registers interactions of individual photons into one or more energy bins. It should be understood that the methods described herein may also be implemented using an energy integration detector.

An acquired projection data set may be used for base material decomposition (BMD). During the BMD, the measured projection is converted to a set of material density projections. The material density projections may be reconstructed to form one pair or a set of material density maps or images (such as bone, soft tissue, and/or contrast agent maps) of each corresponding base material. The density maps or images may then be associated to form volume rendering of a base material (e.g., bone, soft tissue, and/or a contrast agent) in an imaging volume.

Once reconstructed, a base material image produced by the imaging system 200 displays internal features of the examination subject 204 represented by the densities of two base materials. The density images can be displayed to demonstrate the foregoing features. In a conventional method for diagnosing medical conditions (such as disease states), and more generally for diagnosing medical events, a radiologist or physician considers a hard copy or display of a density image to discern characteristic features of interest. Such features may include a lesion, size, and shape of a particular anatomical structure or organ, and other features should be discernible in the image on the basis of the skill and knowledge of an individual practitioner.

In one implementation, the imaging system 200 includes a control mechanism 208 to control movement of the components, such as the rotation of the gantry 102 and the operation of the X-ray source 104. In certain implementations, the control mechanism 208 further includes an X-ray controller 210, configured to provide power and timing signals to the X-ray source 104. Additionally, the control mechanism 208 includes a gantry motor controller 212, configured to control the rotational speed and/or position of the gantry 102 on the basis of imaging requirements.

In certain implementations, the control mechanism 208 further includes a data acquisition system (DAS) 214, and the DAS is configured to sample analog data received from the detector elements 202, and convert the analog data to a digital signal for subsequent processing. The DAS 214 may further be configured to selectively aggregate analog data from a subset of the detector elements 202 into a so-called macro detector, as described further herein. The data sampled and digitized by the DAS 214 is transmitted to a computer or computing device 216. In an example, the computing device 216 stores data in a storage device or large-capacity storage apparatus 218. For example, the storage device 218 may include a hard disk drive, a floppy disk drive, a compact disc-read/write (CD-R/W) drive, a digital versatile disc (DVD) drive, a flash drive, and/or a solid-state storage drive.

Additionally, the computing device 216 provides commands and parameters to one or more of the DAS 214, the X-ray controller 210, and the gantry motor controller 212 to control system operations, such as data acquisition and/or processing. In certain embodiments, the computing device 216 controls system operations on the basis of operator input. The computing device 216 receives the operator input by means of an operator console 220 that is operably connected to the computing device 216, the operator input including, for example, commands and/or scan parameters. The operator console 220 may include a keyboard (not shown) or a touch screen to allow the operator to specify commands and/or scan parameters.

Although FIG. 2 shows only one operator console 220, more than one operator console may be coupled to the imaging system 200, for example, for inputting or outputting system parameters, requesting examination, mapping data, and/or viewing images. Moreover, in certain implementations, the imaging system 200 may be coupled to, for example, a plurality of displays, printers, workstations, and/or similar devices located locally or remotely within an institution or hospital or in a completely different location via one or more configurable wired and/or wireless networks (such as the Internet and/or a virtual private network, a wireless telephone network, a wireless local area network, a wired local area network, a wireless wide area network, a wired wide area network, etc.).

In one implementation, for example, the imaging system 200 includes or is coupled to a picture archiving and communication system (PACS) 224. In an exemplary implementation, the PACS 224 is further coupled to a remote system (such as a radiology information system or a hospital information system) and/or coupled to an internal or external network (not shown) to allow an operator at a different position to provide commands and parameters and/or obtain access to image data.

The computing device 216 uses operator-provided and/or system-defined commands and parameters to operate a scanning table motor controller 226, the scanning table motor controller is able to control the scanning table 114, and the scanning table may be an electrical scanning table. Specifically, the scanning table motor controller 226 moves the scanning table 114 to properly position the examination subject 204 in the gantry 102 to acquire projection data corresponding to a target volume of the examination subject 204.

As described previously, the DAS 214 samples and digitizes the projection data acquired by the detector elements 202. Subsequently, an image reconstructor 230 uses the sampled and digitized X-ray data to perform high-speed reconstruction. Although the image reconstructor 230 is shown as a separate entity in FIG. 2, in certain implementations, the image reconstructor 230 may form a part of the computing device 216. Alternatively, the image reconstructor 230 may not be present in the imaging system 200, and the computing device 216 may instead perform one or more functions of the image reconstructor 230. In addition, the image reconstructor 230 may be located locally or remotely and may be operably connected to the imaging system 200 by using a wired or wireless network. Specifically, in one exemplary embodiment, computing resources in a “cloud” network cluster may be used for the image reconstructor 230.

In one embodiment, the image reconstructor 230 stores a reconstructed image in the storage device 218. Alternatively, the image reconstructor 230 may transmit the reconstructed image to the computing device 216 to generate usable patient information for diagnosis and evaluation. In certain implementations, the computing device 216 may transmit the reconstructed image and/or patient information to a display or display device 232, the display or display device being communicatively coupled to the computing device 216 and/or the image reconstructor 230. In some implementations, the reconstructed image may be transmitted from the computing device 216 or the image reconstructor 230 to the storage device 218 for short-term or long-term storage.

In CT imaging, an X-ray intensity attenuation formula can be used to quantitatively describe attenuation of the intensity of X-rays when passing through different tissues. The X-ray intensity attenuation formula can be expressed as: I=I₀e^{-μx}. I is the X-ray intensity after the X-ray passes through an absorbing medium. I₀ is an initial X-ray intensity, i.e., the intensity before the X-ray passes through no material. μ is a linear attenuation coefficient of the medium for the X-ray (the unit is usually cm⁻¹), and is related to a tissue type, different tissues having different attenuation coefficients. x is the path length of the X-ray passing through the medium (e.g., the thickness of the absorbing medium). In CT imaging, an X-ray beam emitted by the X-ray source passes through a patient's body, and different tissues absorb the X-ray differently, resulting in different intensities received by a detector. The X-ray intensity at different locations is measured, so that an image of an internal structure can be reconstructed. From the above formula, I₀ represents the initial intensity emitted by the X-ray source, and is the basis of the raw data in CT imaging. If the value of I₀ is inaccurate, measurement of the attenuation coefficient of the tissue in the patient is directly affected, and then final image quality is affected. Therefore, during CT imaging, I₀ usually needs to be calibrated under each scanning condition, to obtain a calibration data set. During image reconstruction, the CT imaging system requires calibration data so as to obtain an accurate image. The calibration data needs to match a system state so as to ensure good image quality. However, the state of the imaging system changes over time, and the calibration data gradually deviates. In practice, the system needs to constantly update the calibration data set by performing a calibration operation (e.g., a daily calibration routine) often for all scanning conditions.

The frequency of the calibration operation is every day or every several days. The calibration operation enables the X-rays, and the X-rays usually affect the service life of an X-ray tube. In addition, the calibration operation is time-consuming. A typical 256-slice CT imaging system is used as an example. Based on the conventional calibration operation logic, scanning is performed about 375 times, and this process takes about 1.5 hours. To reduce the number of times scanning is performed, it is proposed to perform the calibration operation only under some scanning conditions, while calibration data of other scanning conditions is calculated. In such a method, although a calibration data set under all scanning conditions can be obtained, the accuracy of the calibration data cannot be guaranteed, which means that the image quality of the imaging system may still not meet expectations even if the calibration operation has just been completed.

The present disclosure provides a method for medical imaging. The method includes: obtaining a scanning protocol for an examination subject. The method includes: for the scanning protocol, obtaining calibration data associated with the scanning protocol in response to a commonly used scanning protocol calibration data set of a medical imaging system including the calibration data. Alternatively, the method includes: for the scanning protocol, performing a calibration operation in response to a commonly used scanning protocol calibration data set of a medical imaging system not including calibration data associated with the scanning protocol, to generate calibration data associated with the scanning protocol. The method includes: scanning the examination subject based on the scanning protocol, to obtain raw imaging data; and performing reconstruction using the calibration data associated with the scanning protocol and the raw imaging data, to obtain a medical image of a scanned subject. The method in the present disclosure can ensure good imaging quality while reducing the number of times exposure detection is required to be performed for calibration operations, and helps prolong the service life of an X-ray tube and improve the use efficiency of the medical imaging system.

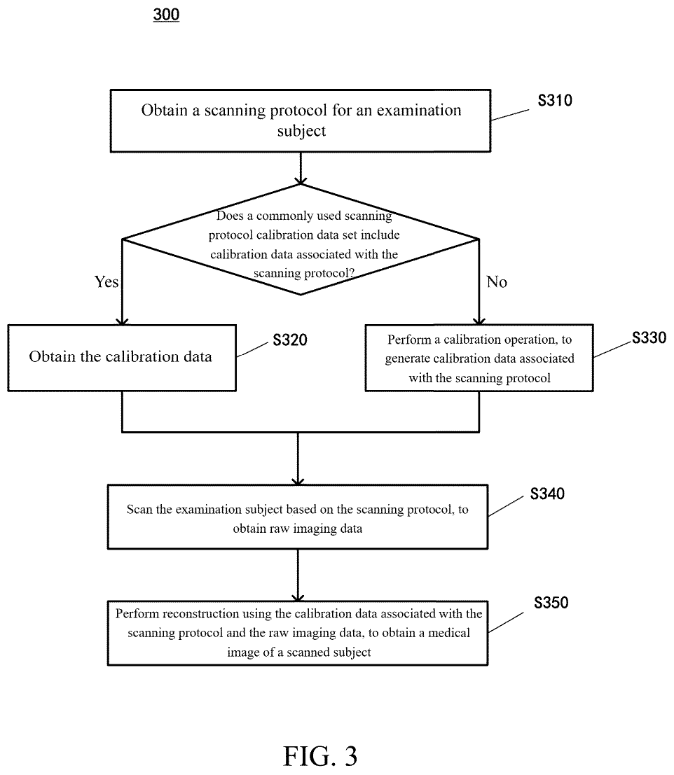

Referring to FIG. 3, FIG. 3 shows a schematic flowchart of a method 300 for medical imaging according to an exemplary embodiment of the present disclosure. In the embodiment, the method 300 is applied to, for example, the imaging system 100 or 200 shown in FIG. 1 or FIG. 2. As shown in FIG. 3, the method 300 for medical imaging according to the embodiment may include the following steps S310 to S350.

In step S310, a scanning protocol for an examination subject is obtained. The scanning protocol may come from a radiology information system (RIS), may be selected from a predefined list stored on the imaging system 100, or may be inputted by a user by using an input unit (e.g., via a keyboard or voice). The scanning protocol may be a description of an imaging examination. For example, the scanning protocol may include may include, for example, the age, sex, weight, body part (such as the kidney, liver, spleen, stomach, adrenal glands, pancreas, colon, or the like), or body region (such as the brain, neck, heart, chest, abdomen, knee, or the like) of a patient, a previous diagnosis, an imaging device type, medical institution-specific applications of an imaging device, layout planning of the imaging device, and/or contrast agent information. The scanning protocol may provide various parameters and related information for performing scans and post-processing, such as a power value, the duration of radiation, speed of movement, radiation energy, and a time delay between image captures, etc. It is conceivable that any configurable technical parameter that should be used for imaging examination by the imaging system 100 may be defined in the scanning protocol. The scanning protocol may be pre-stored in the imaging system 100, for example, in a memory of the computing device 216. The scanning protocol may also be stored in a separate memory or in a remote cloud, and in this case, the scanning protocol may be transmitted to the computing device 216 via a wired or wireless network. The scanning protocol may be provided by a manufacturer of the imaging system 100, or may be predefined by a user of the imaging system 100. The scanning protocol may also be provided by users of medical imaging systems of other medical institutions. In the embodiments of the present disclosure, the scanning protocol includes at least various parameters and related information for scanning and post-processing by the imaging system 100.

In step S320, for the scanning protocol for the examination subject, calibration data associated with the scanning protocol is obtained in response to a commonly used scanning protocol calibration data set of a medical imaging system including the calibration data.

In step S330, for the scanning protocol for the examination subject, a calibration operation is performed in response to a commonly used scanning protocol calibration data set of a medical imaging system not including calibration data associated with the scanning protocol, to generate calibration data associated with the scanning protocol.

The commonly used scanning protocol calibration data set may include calibration data associated with a commonly used scanning protocol. The commonly used scanning protocol may be those scanning protocols that have been frequently used in the past, or will be frequently used in the near future or in the future in the medical imaging system. Each scanning protocol may correspond to one calibration scanning condition. The calibration data set may include a set of calibration data of an X-ray tube under each calibration scanning condition. The calibration data set may be recorded in the form of a table or a vector, or another suitable data form. For example, the calibration scanning condition includes a tube voltage, a collimator aperture width, a X-ray shape filter size, and/or a focal spot (tube focal spot) size. The tube voltage is a high voltage applied to an X-ray tube. When calibration scanning is performed, the tube voltage may include 70 kVp, 80 kVp, 100 kVp, 120 kVp, and 140 kVp. A collimator is located at an outlet for the X-ray of the X-ray tube and before the examination subject, and is used for adjusting the size of a fan-beam or cone-beam X-ray emitted by the X-ray tube. The collimator aperture width is the size of an opening of an X-ray shielding gate of the collimator in a direction (the Z direction shown in FIG. 1) in which the examination subject enters or exits a CT imaging device, i.e., the size in the width direction of an X-ray beam. The CT imaging apparatus adjusts the coverage range of the X-ray tube by adjusting the value of the collimator aperture width. That is, the collimator aperture width represents the coverage range of the X-ray beam emitted by the tube. When calibration scanning is performed, the collimator aperture width may be a plurality of discrete sizes between 5 mm and 160 mm. The X-ray shape filter is used for manipulating and further changing the spectral or spatial intensity distribution of X-ray radiation. For example, a bowtie filter additionally causes X-ray radiation to be focused or expanded by means of a protruding or recessed face. Typically, X-ray shape filters are divided into three types, i.e., large, medium, and small X-ray shape filters. The focal spot size may be divided into three types, i.e., large, medium, and small sizes. In some embodiments, each calibration scanning condition can be a combination of the above four aspects of factors. For example, X-ray tube calibration data is obtained after scanning is performed by the CT imaging device under the calibration scanning condition: the tube voltage is 70 kVp, the collimator aperture width is 5 mm, the before-patient ray filter is large, and the focal spot is small.

In some embodiments, the calibration data may be an initial X-ray intensity I₀. The calibration data set may include the initial X-ray intensity I₀ under a plurality of different calibration scanning conditions. The commonly used scanning protocol calibration data set may include calibration data corresponding to scanning protocols commonly used in the medical imaging system under those calibration scanning conditions.

Referring to FIG. 3, when the commonly used scanning protocol calibration data set of the medical imaging system includes calibration data associated with a scanning protocol for a current examination subject, step S320 may be performed, that is, the calibration data is obtained for subsequent processing, such as image reconstruction. When the commonly used scanning protocol calibration data set of the medical imaging system does not include calibration data associated with a scanning protocol for a current examination subject, step S330 may be performed, that is, a calibration operation may be performed to generate calibration data associated with the scanning protocol for the current examination subject.

In step S340, the examination subject is scanned based on the scanning protocol, to obtain raw imaging data. The raw imaging data may include a group of X-ray radiation attenuation measurement results (e.g., projection data) of the examination subject, or may include pre-processed projection data. In some embodiments, the raw imaging data may include data directly or indirectly represented by the X-ray intensity after an X-ray passes through the examination subject.

In step S350, reconstruction is performed using the calibration data associated with the scanning protocol and the raw imaging data, to obtain a medical image of a scanned subject. The method for medical imaging according to the exemplary embodiment of the present disclosure is described above. In the method, whether the commonly used scanning protocol calibration data set of the medical imaging system includes the calibration data associated with the scanning protocol can be determined after a current scanning protocol is received, and if the commonly used scanning protocol calibration data set of the medical imaging system includes the calibration data associated with the scanning protocol, the calibration data may be directly used to perform image reconstruction. If the commonly used scanning protocol calibration data set of the medical imaging system does not include calibration data associated with the scanning protocol, a targeted calibration operation (single calibration operation) may be performed to generate associated calibration data for image reconstruction. Compared with performing a full calibration operation for all scanning conditions at an interval of a period of time, in the method in the present disclosure, the number of times of exposure to which the X-ray tube is subjected when the full calibration operation is performed can be reduced, and the service life is prolonged. In addition, overall time spent by the medical imaging system on the calibration operation can be reduced, which is beneficial to improving the use efficiency of the medical imaging system.

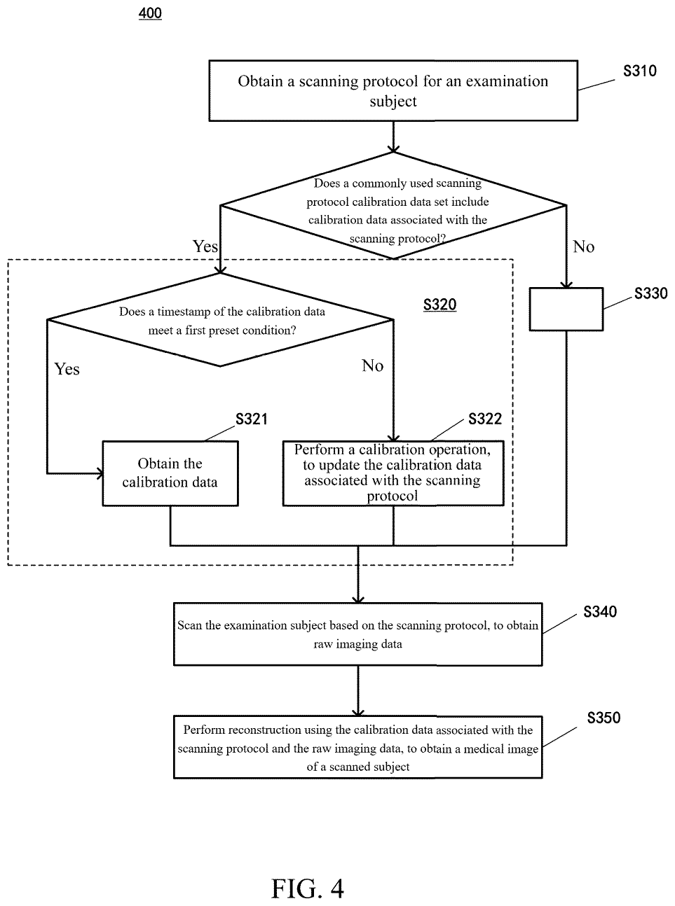

FIG. 4 shows a schematic flowchart of a method 400 for medical imaging according to an optional embodiment of the present disclosure. Some details of the method 400 for medical imaging according to an optional embodiment of the present disclosure are the same as or similar to those of the method 300 for medical imaging according to the exemplary embodiment of the present disclosure. Special features of the method 400 for medical imaging are mainly described below.

In the method 400, step S320 may include sub-steps S321 and S322. In sub-step S321, the calibration data is obtained in response to a timestamp of the calibration data meeting a first preset condition. In sub-step S322, the calibration operation is performed in response to the timestamp of the calibration data not meeting the first preset condition, to update the calibration data associated with the scanning protocol.

The first preset condition may be that an interval between the timestamp of the calibration data associated with the current scanning protocol and a current time is less than or equal to a first threshold. The first threshold may be in the form of a quantity of days, a quantity of weeks, a quantity of months, or another suitable time form. For example, the first threshold may include 2 days, 3 days, 4 days, 5 days, 6 days, 7 days, 14 days, 21 days, 28 days, 56 days, or any other quantity of days. When the interval between the timestamp of the calibration data and the current time is less than or equal to the first threshold, it may be considered that the calibration data is fresh, and a change in the state of the imaging system may be small. Therefore, a deviation between the calibration data and an actual value may be small, which will not cause significant deterioration in the quality of a reconstructed image, and therefore, may be directly used for subsequent processing without performing a calibration operation for the current scanning protocol. When the interval between the timestamp of the calibration data and the current time is greater than the first threshold (that is, the first preset condition is not met), it may be considered that the calibration data is old, and the change in the state of the imaging system may be large. Therefore, the deviation between the calibration data and the actual value may be large, which may cause significant deterioration in the quality of the reconstructed image. Therefore, a calibration operation needs to be performed for the current scanning protocol, to update the calibration data for subsequent processing. In this regard, when the timestamp of the calibration data meets the first preset condition, sub-step S321 may be performed, that is, the calibration data is obtained for subsequent processing, such as image reconstruction. When the timestamp of the calibration data does not satisfy the first preset condition, step S322 may be performed, that is, a calibration operation may be performed to update the calibration data associated with the scanning protocol for the current examination subject.

In some embodiments, the calibration operation may be automatically performed in a medical imaging system. For example, in the method 300 or the method 400, when the medical imaging system determines that the commonly used scanning protocol calibration data set of the medical imaging system does not include calibration data associated with the scanning protocol for the current examination subject, the calibration operation may be automatically performed to generate calibration data associated with the scanning protocol for the current examination subject. For another example, in the method 400, when the medical imaging system determines that the commonly used scanning protocol calibration data set of the medical imaging system includes the calibration data associated with the scanning protocol for the current examination subject, and the timestamp of the calibration data does not meet the first preset condition, the calibration operation may be automatically performed to update the calibration data associated with the scanning protocol for the current examination subject.

In some embodiments, the calibration operation may be performed based on reception of an instruction to perform calibration. The method 300 or the method 400 may further include a prompting step of providing prompt information about whether to perform calibration. For example, step S330 may include the prompting step. The prompt information about whether to perform calibration may be provided first in response to the commonly used scanning protocol calibration data set of the medical imaging system not including calibration data associated with the scanning protocol, and after the instruction to perform calibration is received, the calibration operation is performed to generate calibration data associated with the scanning protocol. For another example, sub-step S322 may include the prompting step. The prompt information about whether to perform calibration may be provided first in response to the timestamp of the calibration data not meeting the first preset condition, and after the instruction to perform calibration is received, the calibration operation is performed to update the calibration data associated with the scanning protocol. The instruction to perform calibration may come from an operator of the medical imaging system.

Optionally, the method 400 may further include: calibration data of which the timestamp does not meet the first preset condition is obtained in response to reception of an instruction not to perform calibration, and the calibration data is directly used for subsequent processing, such as image reconstruction. For example, the prompt information about whether to perform calibration may be provided in response to the timestamp of the calibration data not meeting the first preset condition. In this case, if a user of the medical imaging system decides not to perform the calibration operation and uses the calibration data for subsequent processing, the instruction not to perform calibration may be provided.

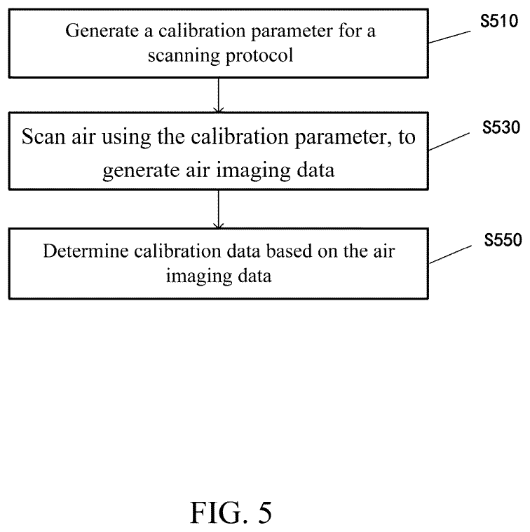

FIG. 5 shows a schematic process of a calibration operation according to an optional embodiment of the present disclosure. In some embodiments, the calibration operation may include air scanning calibration, for example, including sub-steps S510 to S550. In sub-step S510, a calibration parameter for the scanning protocol is generated. The calibration parameter may include at least one of the following parameters: a tube voltage, a collimator aperture width, an X-ray shape filter size, and a focal spot size. In sub-step S530, air is scanned using the calibration parameter, to obtain air imaging data. In sub-step S550, the calibration data is determined based on the air imaging data. When there is no patient or other object, air is scanned using the calibration parameter, to determine the initial X-ray intensity I₀ under the condition of the calibration parameter.

In some embodiments, after the calibration operation is performed, the method 300 or the method 400 may further include: updating the calibration data determined based on the air imaging data, a calibration parameter associated with the calibration data, and a new timestamp in the commonly used scanning protocol calibration data set.

It should be noted that the present disclosure is not intended to limit the calibration operation, and the above air scanning calibration is only illustrated as an example of the calibration operation. Those skilled in the art can understand that existing or future calibration operations can be used to determine the initial X-ray intensity I₀ under the condition of a specific calibration parameter. For example, the calibration operation may include determining the initial X-ray intensity I₀ by measuring the X-ray intensity passing through a phantom using a calibration phantom having a known attenuation characteristic. For another example, the calibration operation may use an automatic exposure control (AEC) system to automatically adjust the X-ray intensity.

In some embodiments, the commonly used scanning protocol calibration data set may include a correspondence between different calibration parameters and calibration data thereof.

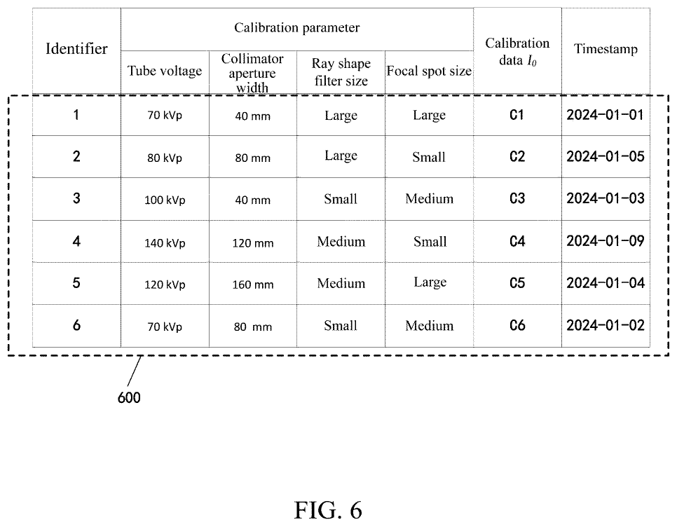

FIG. 6 shows an example of a commonly used scanning protocol calibration data set 600 according to the present disclosure. In this example, the calibration parameter includes a tube voltage, a collimator aperture width, a X-ray shape filter size, and a focal spot size.

In the commonly used scanning protocol calibration data set 600, a calibration parameter group is associated with respective calibration data. In other words, one group of calibration parameters corresponds to one piece of calibration data. The tube voltage may include 70 kVp, 80 kVp, 100 kVp, 120 kVp, 140 kVp, or another value. The collimator aperture width may be any value less than or equal to the maximum coverage of the X-ray tube (i.e., the maximum value of the collimator aperture width). In some examples, the collimator aperture width may be between 5 mm and 160 mm, for example, may be 5 mm, 10 mm, 20 mm, 30 mm, 40 mm, 50 mm, 60 mm, 70 mm, 80 mm, 90 mm, 100 mm, 110 mm, 120 mm, 130 mm, 140 mm, 150 mm, or 160 mm. The X-ray shape filter may include a plurality of types of filters, and the X-ray shape filter size may be generally distinguished as large, medium, and small, but the present disclosure is not limited thereto. The X-ray shape filter size may be divided more finely. For example, different X-ray shape filter sizes are directly represented by using filter names. In some other embodiments, the X-ray shape filter size may alternatively be a scanning field of view (SFOV) of a filter. In other words, the X-ray shape filter size in FIG. 6 may be represented by the SFOV, instead of the representation of “large,” “medium,” and “small.” For example, the X-ray shape filter size may be represented by an angle value, which may include, for example, 5°, 10°, 20°, 30°, 40°, 50°, 60°, or 70°. The focal spot size may be divided into three types, i.e., large, medium, and small sizes. In this example, each calibration scanning condition is a combination of the above four aspects of parameters. For example, a first group of data is X-ray tube calibration data obtained after scanning is performed by the CT imaging device under the calibration scanning condition: the tube voltage is 70 kVp, the collimator aperture width is 40 mm, the filter size is large, and the focal spot is large.

It should be noted that the present disclosure is not intended to limit the calibration parameter group. The above calibration parameter including the tube voltage, the collimator aperture width, the X-ray shape filter size, and the focal spot size is only used as an example for description. Those skilled in the art can understand that the calibration parameter may include one or more, but not all, of the tube voltage, the collimator aperture width, the X-ray shape filter size, or the focal spot size. Alternatively, in another embodiment, the calibration parameter may further include another parameter.

The calibration data set 600 may further include a timestamp for each piece of calibration data, e.g., a generation time or an update time of the calibration data. The timestamp of the calibration data may represent a time of the last calibration operation performed under the condition of a calibration parameter associated with the calibration data, and may be used to represent the freshness of the calibration data.

The calibration data set 600 may further include the number of times of use (not shown) of each piece of calibration data, for example, the number of times of use within a period of time (e.g., a week, two weeks, three weeks, four weeks, or a longer time).

The calibration data set 600 may further include an identifier. The identifier may be used to identify different groups of calibration parameters, to facilitate operations such as retrieving, adding data, deleting data, etc. Although FIG. 6 shows an identifier in the form of a digit, the identifier may be in another form. For example, the identifier may be in the form of a letter, a word, a digit, or a combination thereof.

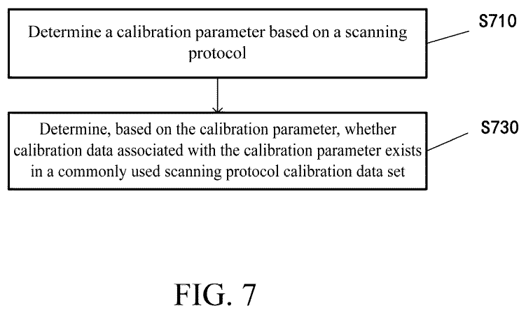

FIG. 7 shows a schematic process of determining whether calibration data exists in a commonly used scanning protocol calibration data set according to the present disclosure. In some embodiments, whether the calibration data associated with the scanning protocol exists in the commonly used scanning protocol calibration data set may be determined via sub-steps S710 and S730.

In sub-step S710, a calibration parameter is determined based on the scanning protocol. As described above, the scanning protocol includes at least various parameters and related information for scanning and post-processing by the imaging system 100. Therefore, the calibration parameter may be determined from the scanning protocol for the current examination subject. For example, the calibration parameter includes at least one of the tube voltage, the collimator aperture width, the X-ray shape filter size, and the focal spot size.

In sub-step S730, whether the calibration data associated with the calibration parameter exists in the commonly used scanning protocol calibration data set is determined based on the calibration parameter. The calibration data associated with the calibration parameter may be retrieved in the commonly used scanning protocol calibration data set 600 shown in FIG. 6 based on the calibration parameter determined in sub-step S710. For example, if the calibration parameters determined in sub-step S710 are a tube voltage of 140 kVp, a collimator aperture width of 120 mm, a medium filter size, and a small focal spot, it may be determined that calibration data C4 associated with this group of calibration parameters exists in the commonly used scanning protocol calibration data set 600.

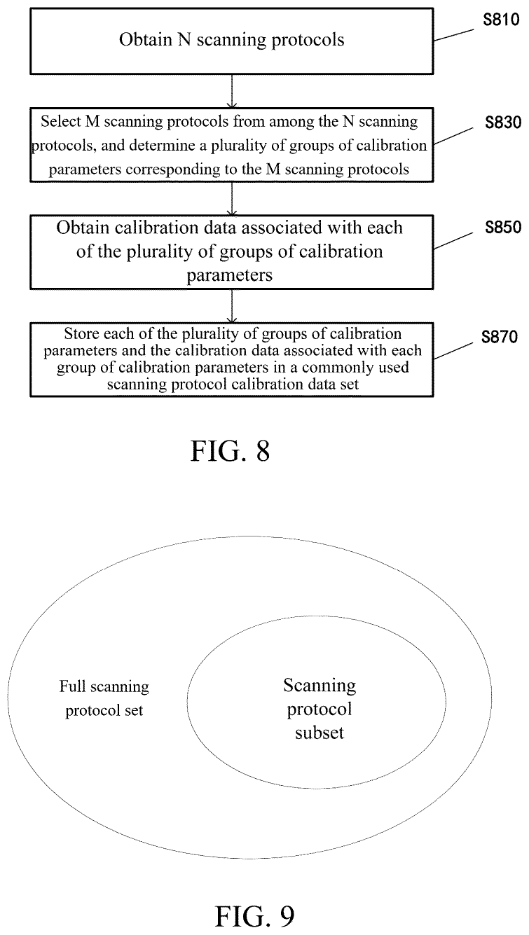

FIG. 8 shows a schematic process of establishing a commonly used scanning protocol calibration data set according to the present disclosure. In some embodiments, the commonly used scanning protocol calibration data set may be established via steps S810 to S870.

In step S810, N scanning protocols are obtained. N is an integer greater than 1. In some embodiments, the N scanning protocols may correspond to a full scanning protocol set (see FIG. 9) of the medical imaging system. For example, a manufacturer of the medical imaging system may provide the full scanning protocol set that may be scanned by the medical imaging system.

In step S830, M scanning protocols are selected from among the N scanning protocols, and a plurality of groups of calibration parameters corresponding to the M scanning protocols are determined. N is greater than M, and M is an integer greater than 0. In some embodiments, the M scanning protocols may correspond to a scanning protocol subset of the medical imaging system (see FIG. 9). For example, the user of the medical imaging system may select M commonly used scanning protocols from the full scanning protocol set. Each scanning protocol has a corresponding calibration parameter. It can be understood that different scanning protocols may have the same calibration parameter. Therefore, the quantity of groups of calibration parameters corresponding to the commonly used M scanning protocols is not necessarily equal to M, but may be less than M. Each group of calibration parameters may include at least one of the tube voltage, the collimator aperture width, the X-ray shape filter size, and the focal spot size.

In some embodiments, at least a part of the M scanning protocols may be determined based on the number of times of use of a plurality of scanning protocols corresponding to a plurality of times of scanning and imaging performed in a period of time. For example, specific scanning protocols that are commonly used scanning protocols may be determined based on the number of times of use of a plurality of scanning protocols executed by the medical imaging system in a period of time (e.g., one week, two weeks, three weeks, four weeks, or a longer time). Specifically, if the M scanning protocols are used to perform imaging more than a specific number of times (e.g., 5 times, 10 times, 15 times, 20 times, 25 times, 30 times, 35 times, or more times) within one month, the M scanning protocols may be considered commonly used scanning protocols, and therefore, the plurality of groups of calibration parameters corresponding to the M scanning protocols are selected.

In some other embodiments, at least a part of the M scanning protocols may be determined based on a plurality of scanning protocols corresponding to a type of a medical institution. For example, the type of the medical institution may include an orthopedic hospital, a pediatric hospital, a chest hospital, a dental hospital, etc. Specifically, if a medical institution in which the medical imaging system is located is a pediatric hospital, it may be considered that M scanning protocols associated with pediatrics are commonly used scanning protocols, and therefore, a plurality of groups of calibration parameters corresponding to the M scanning protocols are selected.

In step S850, calibration data associated with each of the plurality of groups of calibration parameters is obtained. In some embodiments, for each group of calibration parameters, default calibration data associated with the group of calibration parameters may be obtained. The default calibration data may be provided by the manufacturer of the medical imaging system, e.g., stored in a memory of the medical imaging system. In some other embodiments, for each group of calibration parameters, a calibration operation may be performed, to generate calibration data associated with the group of calibration parameters. As an example, the calibration operation may include scanning air using the group of calibration parameters, to obtain air imaging data; and determining the calibration data associated with the group of calibration parameters based on the air imaging data.

In step S870, each of the plurality of groups of calibration parameters and the calibration data associated with each group of calibration parameters are stored in the commonly used scanning protocol calibration data set. For example, when each group of calibration parameters includes a tube voltage, a collimator aperture width, an X-ray shape filter size, and a focal spot size, each group of calibration parameters and calibration data I₀ associated with each group of calibration parameters may be stored in the commonly used scanning protocol calibration data set 600 shown in FIG. 6.

Optionally, the method 300 or 400 for medical imaging may further include: deleting calibration data from the commonly used scanning protocol calibration data set, to refine the commonly used scanning protocol calibration data set. Deletion of the calibration data may be determined based on the timestamp, the number of times of use thereof, or a combination of these two items.

In some embodiments, calibration data of which the number of times of use is less than a second threshold may be deleted from the commonly used scanning protocol calibration data set. The second threshold may be any number of times, for example, 2 times, 5 times, 10 times, 15 times, 20 times, 25 times, 30 times, or another number of times. If the number of times of use of calibration data is small (that is, less than the second threshold), it may be considered that a scanning protocol corresponding to the calibration data is not commonly used, and therefore, the calibration data and information (for example, the calibration parameter, the identifier, and the timestamp) associated with the calibration data may be deleted from the commonly used scanning protocol calibration data set.

In some other embodiments, calibration data of which the timestamp meets a second preset condition may be deleted from the commonly used scanning protocol calibration data set. The second preset condition may include the interval between the timestamp of the calibration data and the current time being greater than or equal to a third threshold. For example, the third threshold may include 7 days, 14 days, 21 days, 28 days, 56 days, or any other quantity of days. The third threshold may be set to be greater than the first threshold described above. When the interval between the timestamp of the calibration data and the current time is greater than or equal to the third threshold, it may be considered that the calibration data has not been used for a long time and is old, and the change in the state of the imaging system may be large. Therefore, the deviation between the calibration data and the actual value may be large, which may cause deterioration in the quality of the reconstructed image. Therefore, the calibration data may be deleted from the commonly used scanning protocol calibration data set.

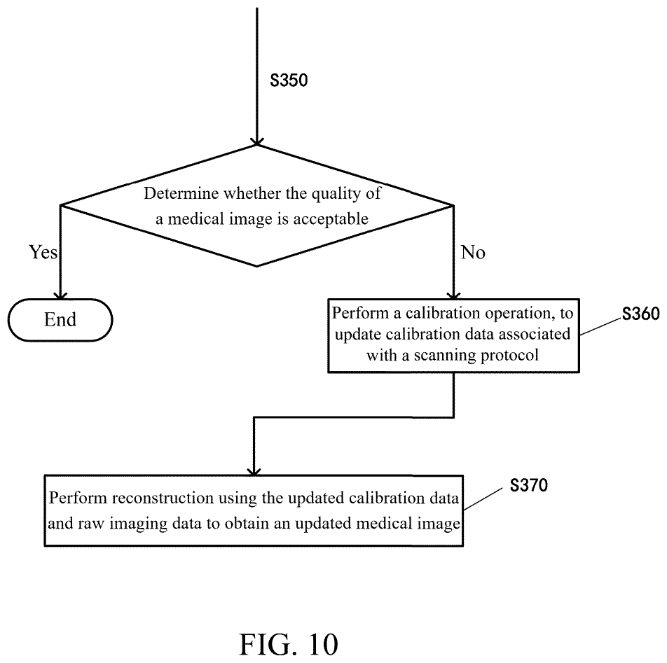

Optionally, the method 300 or 400 for medical imaging may further include steps S360 and S370, as shown in FIG. 10. In the method 300 or 400 for medical imaging, reconstruction may be performed using the calibration data included in the commonly used scanning protocol calibration data set, to obtain the medical image of the scanned subject, or reconstruction may be performed using the calibration data of which the timestamp does not meet the first preset condition according to an instruction of the user, to obtain the medical image of the scanned subject. In both cases, the change in the state of the imaging system may be large, resulting in a large deviation between the calibration data and the actual value, which may cause deterioration in the quality of the reconstructed image. In this regard, after step S350, whether the quality of the medical image is acceptable may be determined. If the quality of the medical image is acceptable, for example, the quality is sufficient for diagnosing the examination subject, the method 300 or 400 may end. If the quality of the medical image is not acceptable, step S360 may be performed. To be specific, a calibration operation is performed to update the calibration data associated with the scanning protocol. As an example, the calibration operation may include generating a calibration parameter for the current scanning protocol; scanning air using the calibration parameter, to obtain air imaging data; and updating the calibration data based on the air imaging data.

After step S360, step S370 may be performed. To be specific, reconstruction is performed using the updated calibration data and the raw imaging data, to obtain an updated medical image. In this way, the quality of the reconstructed image when there is a large deviation between the original calibration data and the actual value can be improved.

In some embodiments, the method 300 or 400 for medical imaging may further include: updating the updated calibration data in step S360, a calibration parameter associated with the updated calibration data, and a new timestamp in the commonly used scanning protocol calibration data set.

According to an exemplary embodiment of the present disclosure, a computer-readable storage medium is further provided. The computer-readable storage medium stores a computer program. The program, when executed by a processor, implements the steps of the method 300 or 400 for medical imaging described above.

According to an exemplary embodiment of the present disclosure, a computer program product is further provided. The computer program product includes instructions. The instructions can be performed by a processor, to implement the method 300 or 400 for medical imaging described above.

According to an exemplary embodiment of the present disclosure, a medical imaging system is further provided. The medical imaging system (for example, the imaging system 100 or 200 in FIG. 1 or FIG. 2) may include a medical imaging device configured to perform scanning and imaging on a scanned subject, and a computer. The computer may be configured to perform the method 300 or 400 for medical imaging described above.

One or a plurality of the above-described techniques and/or embodiments may be implemented using hardware and/or software or include hardware and/or software, for example, modules or apparatuses executed on one or a plurality of computing devices 216. Of course, the modules or apparatuses described herein show various functions and are not limited to limiting the structure and functions of any embodiment. On the contrary, the functions of various modules or apparatuses may be divided and executed differently according to more or fewer modules or apparatuses considered by various designs.

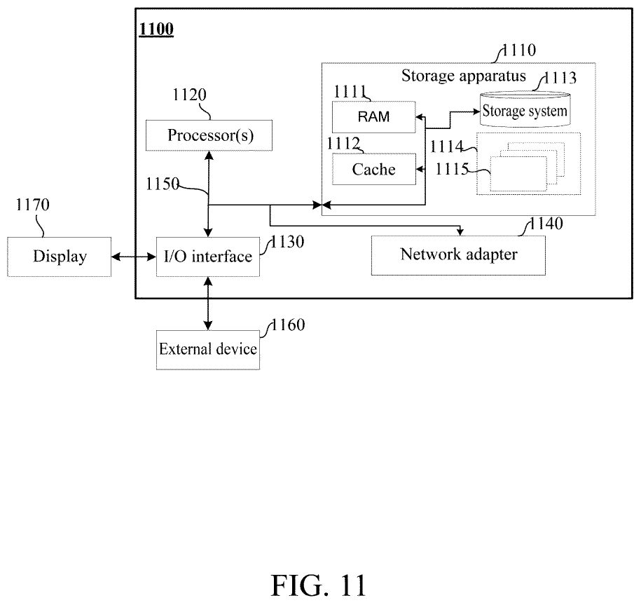

FIG. 11 shows an example of an electronic device 1100 according to an embodiment of the present disclosure. The electronic device 1100 includes: one or more processors 1120; and a storage apparatus 1110, configured to store one or more programs, wherein when the one or more programs are executed by the one or more processors 1120, and the one or more processors 1120 are enabled to implement the method for medical imaging provided in the embodiments of the present disclosure. The processor is, for example, a digital signal processor (DSP), a microcontroller, an application-specific integrated circuit (ASIC), or a microprocessor.

The electronic device 1100 shown in FIG. 11 is merely an example, and should not cause any limitation to the function and use scope of the embodiments of the present disclosure. As shown in FIG. 11, the electronic device 1100 is represented in the form of a general-purpose computing device. Components of the electronic device 1100 may include, but are not limited to: one or more processors 1120, a storage apparatus 1110, and a bus 1150 connecting different system components (including the storage apparatus 1110 and the processor 1120).

The bus 1150 represents one or more of several types of bus structures, including a memory bus or a memory controller, a peripheral bus, an accelerated graphics port, a processor, or a local bus using any of a plurality of bus structures. For example, these architectures include, but are not limited to, an Industrial Standard Architecture (ISA) bus, a Micro Channel Architecture (MAC) bus, an enhanced ISA bus, a Video Electronics Standards Association (VESA) local bus, and a Peripheral Component Interconnect (PCI) bus.

The electronic device 1100 typically includes a plurality of computer system readable media. These media may be any available media that can be accessed by the electronic device 1100, including volatile and non-volatile media as well as removable and non-removable media.

The storage apparatus 1110 may include a computer system-readable medium in the form of a volatile memory, for example, a random access memory (RAM) 1111 and/or a cache memory 1112. The electronic device 1100 may further include other removable/non-removable, and volatile/non-volatile computer system storage media. Only as an example, a storage system 1113 may be configured to read/write a non-removable, non-volatile magnetic medium (not shown in FIG. 11, typically referred to as a “hard disk drive”). Although not shown in FIG. 11, a magnetic disk drive configured to read/write a removable non-volatile magnetic disk (for example, a “floppy disk”) and an optical disc drive configured to read/write a removable non-volatile optical disc (for example, a CD-ROM, a DVD-ROM, or other optical media) may be provided. In these cases, each drive may be connected to the bus 1150 via one or more data medium interfaces. The storage apparatus 1110 may include at least one program product which has a group of program modules (for example, at least one program module) configured to execute the functions of the embodiments of the present disclosure.

A program/utility tool 1114 having a group (at least one) of program modules 1115 may be stored in, for example, the storage apparatus 1110. This program module 1115 includes, but is not limited to, an operating system, one or more applications, other program modules, and program data, and each of these examples or a certain combination thereof may include an implementation of a network environment. The program module 1115 typically executes the function and/or method in any embodiment described in the present disclosure.

The electronic device 1100 may also communicate with one or more external devices 1160 (such as a keyboard, a pointing device, and a display 1170), and may further communicate with one or more devices that enable a user to interact with the electronic device 1100, and/or communicate with any device (such as a network card and a modem) that enables the electronic device 1100 to communicate with one or more other computing devices. Such communication may be carried out via an input/output (I/O) interface 1130. In addition, the electronic device 1100 may also communicate with one or more networks (for example, a local area network (LAN), a wide area network (WAN), and/or a public network, such as the Internet) via a network adapter 1140. As shown in FIG. 11, the network adapter 1140 communicates with other modules of the electronic device 1100 through the bus 1150. It should be understood that although not shown in the drawing, other hardware and/or software modules may be used in conjunction with the electronic device 1100, the modules including, but not being limited to: microcode, device drivers, redundant processing units, external disk drive arrays, RAID systems, tape drives, data backup storage systems, and the like. The processor 1120, by running programs stored in the storage apparatus 1110, executes various functional applications and data processing, such as implementing the method provided by the embodiments of the present disclosure.

The technique described herein may be implemented with hardware, software, firmware, or any combination thereof, unless specifically described as being implemented in a specific manner. Any features described as modules or components may also be implemented together in an integrated logical device, or separately implemented as discrete but interoperable logical devices. If implemented with software, the technique may be implemented at least in part by a non-transitory processor-readable storage medium that includes instructions, wherein when executed, the instructions perform one or more of the aforementioned methods. The non-transitory processor-readable data storage medium may form part of a computer program product that may include an encapsulation material. Program code may be implemented in a high-level procedural programming language or an object-oriented programming language so as to communicate with a processing system. If desired, the program code may also be implemented in an assembly language or a machine language. In fact, the mechanisms described herein are not limited to the scope of any particular programming language. In any case, the language may be a compiled language or an interpreted language.

One or a plurality of aspects of at least some embodiments may be implemented by representative instructions that are stored in a machine-readable medium and represent various logic in a processor, wherein when read by a machine, the representative instructions cause the machine to manufacture the logic for executing the technique described herein.

Such machine-readable storage media may include, but are not limited to, a non-transitory tangible arrangement of an article manufactured or formed by a machine or device, including storage media, such as: a hard disk; any other types of disk, including a floppy disk, an optical disk, a compact disk read-only memory (CD-ROM), compact disk rewritable (CD-RW), and a magneto-optical disk; a semiconductor device such as a read-only memory (ROM), a random access memory (RAM) such as a dynamic random access memory (DRAM) and a static random access memory (SRAM), an erasable programmable read-only memory (EPROM), a flash memory, and an electrically erasable programmable read-only memory (EEPROM); a phase change memory (PCM); a magnetic or optical card; or any other type of medium suitable for storing electronic instructions.

Instructions may further be sent or received by means of a network interface device that uses any of a number of transport protocols (for example, Frame Relay, Internet Protocol (IP), Transfer Control Protocol (TCP), User Datagram Protocol (UDP), and Hypertext Transfer Protocol (HTTP)) and through a communication network using a transmission medium.

An exemplary communication network may include a local area network (LAN), a wide area network (WAN), a packet data network (for example, the Internet), a mobile phone network (for example, a cellular network), a plain old telephone service (POTS) network, and a wireless data network (for example, Institute of Electrical and Electronics Engineers (IEEE) 802.11 standards referred to as Wi-Fi®, and IEEE 802.19 standards referred to as WiMax®), IEEE 802.15.4 standards, a peer-to-peer (P2P) network, and the like. In an example, the network interface device may include one or a plurality of physical jacks (for example, Ethernet, coaxial, or phone jacks) or one or a plurality of antennas for connection to the communication network. In an example, the network interface device may include a plurality of antennas that wirelessly communicate using at least one technique among single-input multiple-output (SIMO), multiple-input multiple-output (MIMO), or multiple-input single-output (MISO) techniques.

The term “transmission medium” should be considered to include any intangible medium capable of storing, encoding, or carrying instructions for execution by a machine, and the “transmission medium” includes digital or analog communication signals or any other intangible medium for facilitating communication of such software.

So far, the method for medical imaging and the medical imaging system according to the present disclosure have been described, and the computer-readable storage medium and the computer program product that can implement the method have been further described.

In the method in the present disclosure, whether the commonly used scanning protocol calibration data set of the medical imaging system includes calibration data associated with the scanning protocol can be determined after the current scanning protocol is received, and if the commonly used scanning protocol calibration data set of the medical imaging system includes the calibration data associated with the scanning protocol, the calibration data may be directly used to perform image reconstruction. If the commonly used scanning protocol calibration data set of the medical imaging system does not include calibration data associated with the scanning protocol, a targeted calibration operation (single calibration operation) may be performed to generate associated calibration data for image reconstruction. Compared with performing a full calibration operation for all scanning conditions at an interval of a period of time, in the method in the present disclosure, the number of times of exposure to which the X-ray tube is subjected when the full calibration operation is performed can be reduced, and the service life is prolonged. In addition, overall time spent by the medical imaging system on the calibration operation can be reduced, which is beneficial to improving the use efficiency of the medical imaging system.

Furthermore, the technique in the present disclosure may be implemented as a fully automatic procedure and is fast and intelligent, without the need for human or perceptual determination.

Some exemplary embodiments have been described above. However, it should be understood that various modifications can be made to the exemplary embodiments described above without departing from the spirit and scope of the present disclosure. For example, an appropriate result can be achieved if the described techniques are performed in a different order and/or if the components of the described system, architecture, device, or circuit are combined in a different manner and/or replaced or supplemented with additional components or equivalents thereof. Accordingly, the modified other embodiments also fall within the protection scope of the claims.

1. A method for imaging comprising:
obtaining a scanning protocol for an examination subject, wherein the scanning protocol includes:
obtaining calibration data associated with the scanning protocol in response to a commonly used scanning protocol calibration data set of an imaging system including the calibration data; or
performing a calibration operation in response to a commonly used scanning protocol calibration data set of the imaging system not including calibration data associated with the scanning protocol, to generate calibration data associated with the scanning protocol;
scanning the examination subject based on the scanning protocol to obtain raw imaging data; and
performing reconstruction using the calibration data associated with the scanning protocol and the raw imaging data to obtain an image of the scanned subject.

2. The method according to claim 1, further comprising obtaining the calibration data in response to a timestamp of the calibration data meeting a first preset condition.

3. The method according to claim 2, further comprising performing the calibration operation in response to the timestamp of the calibration data not meeting the first preset condition to update the calibration data associated with the scanning protocol.

4. The method according to claim 1, wherein the calibration operation is automatically performed by the imaging system.

5. The method according to claim 1, wherein the calibration operation is performed based on reception of an instruction to perform calibration, and the method further includes providing prompt information about whether to perform calibration.

6. The method according to claim 1, wherein the calibration operation includes:
generating a calibration parameter for the scanning protocol;
scanning air using the calibration parameter to obtain air imaging data; and
determining the calibration data based on the air imaging data.

7. The method according to claim 6, further including updating the calibration data determined based on the air imaging data, a calibration parameter associated with the calibration data, and a new timestamp in the commonly used scanning protocol calibration data set.

8. The method according to claim 1, wherein the commonly used scanning protocol calibration data set includes a correspondence between different calibration parameters and calibration data thereof; and
determining whether the calibration data associated with the scanning protocol exists in the commonly used scanning protocol calibration data set includes:
determining a calibration parameter based on the scanning protocol; and
determining, based on the calibration parameter, whether the calibration data associated with the calibration parameter exists in the commonly used scanning protocol calibration data set.

9. The method according to claim 8, wherein the commonly used scanning protocol calibration data set is generated via the following steps:
obtaining N scanning protocols, wherein N is an integer greater than 1;
selecting M scanning protocols from among the N scanning protocols, and determining a plurality of groups of calibration parameters corresponding to the M scanning protocols, wherein N is greater than M, and M is an integer greater than 0;
obtaining calibration data associated with each of the plurality of groups of calibration parameters; and
storing each of the plurality of groups of calibration parameters and the calibration data associated with each group of calibration parameters in the commonly used scanning protocol calibration data set.

10. The method according to claim 9, wherein the selecting M scanning protocols from among the N scanning protocols includes:
determining at least a part of the M scanning protocols based on the number of times of use of a plurality of scanning protocols corresponding to a plurality of times of scanning and imaging performed in a period of time; and/or
determining at least a part of the M scanning protocols based on a plurality of scanning protocols corresponding to a type of a medical institution.

11. The method according to claim 9, wherein the obtaining calibration data associated with each of the plurality of groups of calibration parameters includes:
for each group of calibration parameters:
obtaining default calibration data associated with the group of calibration parameters; or
performing a calibration operation, to generate calibration data associated with the group of calibration parameters.

12. The method according to claim 11, wherein the calibration operation includes:
scanning air using the group of calibration parameters, to obtain air imaging data; and
determining the calibration data associated with the group of calibration parameters based on the air imaging data.

13. The method according to claim 6, wherein the calibration parameter includes at least one of the following: a tube voltage, a collimator aperture width, an X-ray shape filter size, and a focal spot size.

14. The method according to claim 2, wherein the first preset condition includes an interval between the timestamp and a current time being less than or equal to a first threshold.

15. The method according to claim 2, further comprising:
deleting from the commonly used scanning protocol calibration data set calibration data of which a timestamp meets a second preset condition; and/or
deleting from the commonly used scanning protocol calibration data set calibration data of which the number of times of use is less than a second threshold.

16. The method according to claim 15, wherein the second preset condition includes an interval between the timestamp and a current time being greater than or equal to a third threshold.

17. The method according to claim 1, further comprising:
determining whether the quality of the medical image is acceptable;
performing a calibration operation in response to determining that the quality of the medical image is unacceptable, to update calibration data associated with the calibration parameter; and
performing reconstruction using the updated calibration data and the raw imaging data, to obtain an updated medical image.

18. The method according to claim 17, wherein the calibration operation comprises:
generating a calibration parameter for the scanning protocol;
scanning air using the calibration parameter, to obtain air imaging data; and
updating the calibration data based on the air imaging data.

19. The method according to claim 17, further including updating the updated calibration data, a calibration parameter associated with the updated calibration data, and a new timestamp in the commonly used scanning protocol calibration data set.
